(19) United States
(12) Patent Application Publication
(10) Pub. No.: US 2005/0118256 A1
(43) Pub. Date: Jun. 2, 2005

(54) Extended release alpha-2 agonist pharmaceutical dosage forms

(76) Inventors: Nilendu Sen, Navi Mumbai (IN); Kavita Chandurkar, St. John's (CA); Anandi Krishnan, Vashi (IN)

Correspondence Address: DILWORTH & BARRESE, LLP, 333 EARLE OVINGTON BLVD., UNIONDALE, NY, 11553, US

(21) Appl. No.: 10/987,750

(22) Filed: Nov. 12, 2004

Related U.S. Application Data
(60) Provisional application No. 60/549,477, filed on Mar. 2, 2004.

(30) Foreign Application Priority Data
Nov. 12, 2003 (IN) 1180/MUM/2003

(51) Int. Cl. A61K 9/48; A61K 9/22; A61K 31/433

(52) U.S. Cl. 424/451; 424/468; 514/362

(57) ABSTRACT

Disclosed is an extended release pharmaceutical formulation containing at least an alpha-2 adrenergic agonist, such as tizanidine, for the treatment and prevention of spasticity in a subject, e.g., painful inflammatory conditions associated with skeletal muscle spasms.

### PRIORITY

This application claims priority under 35 U.S.C. §119 to U.S. Provisional Application No. 60/549,477, filed Mar. 2, 2004, and from Indian Provisional Application No.1180/MUM/2003, filed Nov. 12, 2003, the contents of which are incorporated by reference herein.

### BACKGROUND OF THE INVENTION

1. Field of the Invention

The present invention relates generally to extended release oral pharmaceutical formulations containing at least an alpha-2 adrenergic agonist which acts as a muscle relaxant and is used in the treatment and management of spasticity.

2. Description of the Related Art

Traditional drug delivery systems include solid oral pharmaceutical dosage forms which are comprised of immediate release (IR) dosages in the form of tablets or capsules. These IR dosage forms release the active drug substance into the body of a subject at a rate that can initially be very high followed by a rapid decline. One potential result of an IR dosage form is that the subject may have varying degrees of blood level fluctuation, which may result in transient therapeutic overdose, followed by a period of therapeutic under dosing. These blood level fluctuations are known as “peaks” and “valleys” or “peaks” and “troughs.”

One of the most frequently utilized methods to extend the duration of drug action in the body and/or control blood level fluctuations is modification of the pharmaceutical dosage form. This is usually achieved with single or multi-component matrix systems in such forms as, for example, granules, pellets, tablets or a combination of the above, where the drug delivery is mainly controlled by a diffusion, osmotic or erosion mechanism.

Extended release formulations have the advantage that the active drug substance is gradually released over a relatively long period so that the drug can be maintained in the blood stream for a longer period of time. The extended release formulations may also keep the drug in the blood stream at a more uniform concentration than would otherwise be the case. In this manner, the formulation can be administered only once or twice daily for drugs that would otherwise have to be taken more frequently to maintain required blood levels. Many different types of extended release oral dosage forms have been developed, but each has disadvantages, which affect its suitability to a particular drug and therapeutic objective.

Alpha adrenoreceptor agonists play an important role in the treatment of pain by blocking nerve impulses. They may also act as skeletal muscle relaxants and can be used in combination with certain anti-inflammatory and analgesic drugs to relieve pain and also give a relaxant effect in certain arthritic conditions. Alpha adrenoreceptor agonists may be used as an active drug substance.

Tizanidine hydrochloride (also known as 5-chloro-4-(2-imidazolin-2-ylamino)-2,1,3-benzothiodiazole hydrochloride and having a molecular formula of C₉H₈CIN₅S—HCl) is a centrally acting alpha-2 adrenergic agonist. Generally, tizanidine HCl is a white to off-white, fine crystalline powder, odorless or with a faint characteristic odor. Tizanidine is slightly soluble in water and methanol; with the solubility in water decreasing as the pH increases.

Tizanidine is an agonist at alpha-2 adrenergic receptor sites and is believed to reduce spasticity by increasing presynaptic inhibition of motor neurons. In animal models, tizanidine has no direct effect on skeletal muscle fibers or the neuromuscular junction, and no major effect on monosynaptic spinal reflexes. The effects of tizanidine are greatest on polysynaptic pathways. The overall effect of these actions is believed to reduce facilitation of spinal motor neurons.

The imidazoline chemical structure of tizanidine is related to that of the anti-hypertensive drug clonidine and other alpha-2 adrenergic agonists. Pharmacological studies in animals show similarities between the two compounds, but tizanidine was found to have one-tenth to one fiftieth (1/50) of the potency of clonidine in lowering blood

Tizanidine is commercially available as 2 mg and 4 mg oral tablets under the brand name Zanaflex®. Tizanidine is administered in tablet form two to three times a day. This type of multi-dose therapy which subjects the patient to peaks and troughs has the potential for dose related side effects. One of the main side effects of tizanidine IR tablets is sedation which may interfere with daily activities. As such, patients taking tizanidine should be warned about performing activities requiring alertness, for example, when driving a vehicle or operating heavy machinery.

One approach to improving the sedative side effects of tizanidine is described in U.S. Pat. No. 6,455,557 which discloses an immediate release multiparticulate composition of tizanidine that provides reduced somnolence in a patient receiving tizanidine therapy. However, frequent dosing may still lead to reduced patient compliance.

U.S. Pat. No. 5,484,607 is directed to a controlled release system for the alpha-agonist, clonidine. The patent describes the method of preparation of an extended/sustained release matrix dosage form incorporating hydrophilic cellulose ethers as the polymeric agents for extended/sustained release of the active ingredient.

International Publication No. WO 03/005951 describes a controlled release formulation ion of a drug in a core, a cylindrical plug embedded in a core and a coating impermeable to the drug.

Accordingly, there remains a need to develop an extended release pharmaceutical formulation of alpha-2 adrenergic agonists such as tizanidine that provides once daily dosing for effective management of spasticity and pain, an improved side effect profile and increased patient compliance.

### SUMMARY OF THE INVENTION

In accordance with one embodiment of the present invention, an extended release pharmaceutical dosage form is provided comprising (a) a core region comprising a therapeutically effective amount of one or more active ingredients comprising one or more alpha-2 adrenegic agonists or a pharmaceutically acceptable salt or ester thereof; (b) a hydrophilic matrix material integrated with the core region and (c) a controlled release coating on the core region.

In another embodiment of the present invention, a pharmaceutical formulation useful for making an oral extended release dosage form is provided comprising (a) a therapeutically effective amount of an active ingredient comprising tizanidine or a pharmaceutically acceptable salt or ester thereof; and, (b) from about 30 to about 70 percent by weight of a pharmaceutically acceptable hydrophilic matrix for the extended release of the active ingredient, and suitable amounts of one or more therapeutically inert, pharmaceutically acceptable adjunct materials.

In yet another embodiment of the present invention, a process for the preparation of an extended release pharmaceutical dosage form is provided comprising:
(a) preparing a core comprising a therapeutically effective amount of an active ingredient comprising one or more alpha-2 adrenergic agonists or a pharmaceutically acceptable salt or ester thereof;
(b) integrating a hydrophilic matrix material with the core that extends the release of the active ingredient; and
(c) coating the core with a controlled release coating for the extended release of the active ingredient.

In yet another embodiment of the present invention, a method for the treatment or prevention of spasticity in a subject is provided comprising administering to the subject in need of such treatment or prevention a therapeutically effective amount of a once-a-day extended release pharmaceutical dosage form comprising (a) a core region comprising a therapeutically effective amount of an active ingredient comprising one or more alpha-2 adrenergic agonists or a pharmaceutically acceptable salt or ester thereof; (b) a hydrophilic matrix material integrated with the core region and (c) a controlled release coating on the core region.

Extended release can advantageously be achieved by embedding an active ingredient comprising one or more alpha-2 adrenergic agonists, e.g., tizanidine HCl, in a matrix of a hydrophilic polymer capable of providing extended release of the active ingredient. Alternatively, extended release can be advantageously achieved by using controlled release coatings over the active ingredient integrated with the matrix. While wishing to not be bound by theory, it is believed that the hydrophilic matrix swells as soon as it comes in contact with water. The water then permeates through the swollen matrix and dissolves the drug thereby allowing the drug to diffuse out of the matrix over a period of about 14 to about 16 hours.

Another aspect of the present invention is to provide an orally administrable extended release pharmaceutical dosage form that when dosed once daily to a patient in need of therapeutic relief from spasticity associated with, for example, a spinal or cerebral injury, muscle spasms or pain resulting from, for example, arthritic conditions, provides therapeutic relief by releasing the active drug substance in such a manner that requisite blood levels are maintained for a time period sufficient to justify once a day dosing and thus ensure patient compliance while reducing potential side effects.

Definitions

The term “extended release” as used herein means a drug dosage system in which the rate of the drug release is more precisely controlled compared to that of immediate or sustained release products, wherein the drug is delivered from the dosage system at a predictable and predetermined rate within the body of a patient such that a therapeutically effective blood level, devoid of peak and trough fluctuations, is maintained over an extended period of time.

The term “drug delivery systems” as used herein means the technology utilized to present the drug to the desired body site for drug release and absorption.

The term “treating” or “treatment” of a state, disorder or condition as used herein shall be understood to mean: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished by, e.g., local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include, but are not limited to, the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include, but are not limited to, the sodium, calcium, potassium and magnesium salts, and the like.

The term “subject” or “a patient” or “a host” as used herein refers to mammalian animals, preferably human.

As used herein the term “antioxidant” is intended to mean an agent which inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluene, hypophosphororus acid, monothioglycerol, propyl gallate, sodium ascorbate, sodium bisulfite, sodium formaldehyde sulfoxylate, sodium metalbisulfite and other such materials known to those of ordinary skill in the art.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol sucrose, fructose and other such materials known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include, but are not limited to, starch, poly(ethylene glycol), guar gum, polysaccharide, bentonites, sugars, invert sugars, poloxamers (e.g., PLURONIC™ F68 and PLURONIC™ f127), collagen, albumin, celluloses in nonaqueous solvents, and the like and combinations thereof. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), and the like and combinations thereof and other such materials known to those of ordinary skill in the art.

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, and the like and combinations thereof and other such materials known to those of ordinary skill in the art.

As used herein, the term “glidant” is intended to mean agents used in tablet and capsule formulations to improve flow-properties during tablet and capsule compression and to produce an anti-caking effect. Such compounds include, by way of example and without limitation, colloidal silica, calcium silicate, magnesium silicate, silicon hydrogel, cornstarch, talc, and the like and combinations thereof and other such materials known to those of ordinary skill in the art.

As used herein, the term “lubricant” is intended to mean substances used in tablet formulations to reduce friction during tablet compression. Such compounds include, by way of example and without limitation, calcium stearate, magnesium stearate, mineral oil, stearic acid, zinc stearate, and the like and combinations thereof and other such materials known to those of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches, e.g., corn starch, potato starch, pre-gelatinized and modified starched thereof; sweeteners; clays, e.g., bentonite, microcrystalline cellulose (e.g. Avicel™), carsium (e.g. Amberlite™), alginates, sodium starch glycolate, gums, e.g., agar, guar, locust bean, karaya, pectin, tragacanth, combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP), tyloxapol (a nonionic liquid polymer of the alkyl aryl polyether alcohol type, also known as superinone or triton), and the like and combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

The present invention relates to extended release pharmaceutical formulations for the treatment or prevention of spasticity in patients suffering from, for example, a spinal or cerebral injury, muscle spasms or pain resulting from, for example, arthritic conditions. It has been found possible to formulate a composition, e.g., as a tablet or capsule, of one or more active ingredients comprising one or more alpha-2 adrenergic agonists, e.g., tizanidine HCl, where the agonist is released in an extended release manner to provide once a day daily dosage. The present invention further provides a composition giving a plasma-concentration profile that proposes to minimize the dose-related side-effects of the drug while maintaining the efficacy within the therapeutic index.

There are several ways of achieving extended release of the alpha-2 adrenergic agonists. In one embodiment, extended release may be achieved by integrating tizanidine or a pharmaceutically acceptable salt or ester thereof with an extended release hydrophilic matrix material and/or coating the core of tizanidine or a pharmaceutically acceptable salt or ester thereof with a controlled release coating. In another embodiment, extended release may be achieved by a controlled release coating over a core comprising one or more agonists integrated in an extended release matrix.

Accordingly, in one embodiment of the present invention, the alpha-2 adrenergic agonists are provided in an extended release formulation, where the extended release is controlled by a controlled release coating in a suitable matrix. The formulation includes at least a core region, which may be, for example, irregular shaped or a collection of mixed shaped granules, and contains at least an active ingredient including at least one or more alpha-2 adrenergic agonists or a pharmaceutically acceptable salt or ester thereof. Suitable alpha-2 adrenergic agonists include, but are not limited to, tizanidine, clonidine, pharmaceutically acceptable salts and esters thereof, and the like and combinations thereof.

The core can be prepared according to known techniques, e.g., extrusion-spheronization (where drug(s) and other additives are granulated by addition of a binder solution. The wet mass is passed through, for example, an extruder equipped with a certain size screen. The extrudates can then be spheronized in a marumerizer. The resulting pellets are dried and sieved for further applications); high-shear granulation (where drug(s) and other additives are dry-mixed and then the mixture is wetted by addition of a binder solution in a high shear-granulator/mixer. The granules can be kneaded after wetting by the combined actions of mixing and milling. The resulting granules or pellets are dried and sieved for further applications); and the like. If desired, one or more pharmaceutically active ingredients or pharmaceutically acceptable excipients can be present in the core region. Examples of pharmaceutically acceptable ingredients or excipients include, but are not limited to, blinders, disintegrant agents, fillers, surfactants, stabilizers, lubricants, glidants, antioxidant and the like and mixtures thereof.

In order to achieve a controlled release coating, the core region can be coated as a whole, or the core region may consist of granules and given varying coats. As one skilled in the art will readily appreciate, the varying coats may be of different thicknesses, while some of the active substance, preferably less than about twenty five percent, may be completely uncoated for immediate release. The granules may then be mixed to achieve a blend of granules with varying coatings. Also, the material of the coating may be varied to achieve a controlled release coating, or a combination of varying coating thicknesses and coating materials may be used. Suitable controlled release coatings include, for example, water-insoluble waxes, water-insoluble polymers, e.g., acrylic resins, and water-insoluble celluloses, e.g., ethyl cellulose.

In one embodiment of the present invention, the controlled release coating is an acrylic resin such as a poly(meth)acrylate copolymer, e.g., materials known under the trade name Eudragit® RSPO and Eudragit® NE 30D (an aqueous dispersion of a neutral copolymer of polymethacrylic acid esters), (available from BASF® of Ludwigshafen, Germany), and belong to the class of methacrylic acid copolymer namely poly(meth)acrylates. Poly(meth)acrylates are synthetic cationic and anionic polymers of dimethylaminoethylmethacrylates, methacrylic acid and methacrylic acid esters in varying ratios. They are anionic in character, based on methacrylic acid and methyl methacrylate, for example, having a ratio of free carboxyl groups to methyl-esterified carboxyl groups of about 1:<3, e.g. around about 1:1 or about 1:2, and with a mean molecular weight of about 135,000. They have solubility in an aqueous media at a pH of about 5.5.

A daily intake of about 2 mg/kg body weight of Eudragit® (equivalent to approximately 150 mg for an average adult) may be regarded as essentially safe in humans.

Ethyl cellulose dispersions e.g., those available as Surelease® (Colorcon® of West Point, Pa.) can also be used as a controlled release coating. It gives consistent, uniform drug release independent of pH.

In order to regulate the rate of release of the active substance, the controlled release coating may also contain water-soluble polymers that act as channeling agents, for example, polyvinylpyrrolidone, water-soluble celluloses, for example, hydroxypropyl methylcellulose or hydroxypropylcellulose, or hydrophilic pore formers, for example, sucrose, sodium chloride or mannitol, plasticizers and the like and mixtures thereof.

In another embodiment of the present invention, the alpha-2 adrenergic agonists are provided in an extended release formulation, where the presence of the active drug substance (i.e., the alpha-2 adrenergic agonists) in the matrix controls the extended release formulation. The active drug substance is integrated into the matrix. The active drug substance may be in granular form and mixed with a matrix material. If desired, the granules may then be mixed with other active drug substance granules that have not been mixed with a matrix material. The granules not mixed with a matrix material will provide immediate release. In the present invention, the active substance will preferably be uniformly distributed in the matrix.

Suitable hydrophilic materials for use as a matrix material include, but are not limited to, polymers, Such as, for example, cellulose ethers, cellulose esters and/or acrylic resins and the like and mixtures thereof. Especially preferred matrix materials include, but are not limited to, ethyl cellulose, hydroxypropyl methylcellulose, hydroxypropylcellulose, hydroxymethylcellulose, poly(meth)acrylic acid and/or the derivatives thereof, such as the salts, amides or esters. Hydrophobic materials, such as, for example, hydrophobic polymers, waxes, fats, long-chain fatty acids, fatty alcohols or corresponding esters or ethers or mixtures thereof can also be used as matrix materials. It is also possible to use mixtures of hydrophilic and hydrophobic materials as an extended release matrix material.

In another embodiment, suitable hydrophilic matrixes include hydroxyalkylsubstituted alkylcelluloses having a viscosity of 15,000 cps to 100,000 cps. In a preferred embodiment of the invention, the matrix is a hydroxypropyl methylcellulose (HPMC) of grades HPMC K15M, HPMC K100M, HPMC K100M CR (available as Methocel® and Ethocel® from Dow of Midland, Mich.). Primary control of drug release is achieved by the Methocel® content, varying the ratio of drug to polymer. As the proportion of hydroxypropyl methylcellulose increases, the release rate is reduced. In the case of less water-soluble drugs, viscosity type offers a secondary control mechanism.

Hydroxypropyl methylcellulose is a very versatile material for the formulation of soluble matrix tablets. HPMC is a widely accepted pharmaceutical excipient and is available in a wide range of molecular weights. Effective control of gel viscosity is easily provided. Hydroxypropyl methylcellulose is primarily used as a tablet binder in film coating and as an extended release tablet matrix. Concentrations of between about 2 and about 5% w/w may be used as a binder in either wet or dry granulation processes. The hydrophilic matrix material may be used at levels of about 10 to about 80% w/w in the composition. In another embodiment, the hydrophilic matrix material may be used at levels of about 30 to about 70% w/w in the composition. In another embodiment, the hydrophilic matrix material may be used at levels of about 40 to about 65% w/w in the composition.

Alternatively, the matrix can be polyethylene oxide polymers (available as Polyox® from Dow of Midland, Mich.), which are non-ionic, high molecular weight water-soluble polymers. Molecular weights range from about 100,000 to about 8,000,000. They meet the requirements of the Food Chemicals Codex, the International Codex Alimentarius and US Pharmacopoeia (USP) or National Formulary (NF). The higher molecular weight grades provide delayed drug release via the hydrophilic matrix approach. Polyox® resins are used as controlled release solid dose matrix systems in the concentrations of about 1 to about 5%. Polyox® resins are very versatile polymers for controlled release applications. Upon exposure to water or gastric juices, they hydrate and swell rapidly to form hydrogels with properties ideally suited for controlled drug-delivery vehicles. No interaction between drug and polymer is to be expected because Polyox® resins are nonionic. In one embodiment of the present invention, the matrix material is Polyox® WSR 301.

Several factors affect the rate of drug release from an extended release polymeric matrix. The physicochemical characteristics of the drug such as, for example, degree of water solubility, molecular weight and the diffusion coefficient from the hydrated matrix, play a very important role in determining the mechanism of drug release. Also, physicochemical characteristics of the diluents added to the matrix affect the rate of drug release.

The compositions of the present invention can then be formed into, for example, tablets or capsules. Tablets containing the compositions according to the present invention may be produced by any standard tabletting technique, e.g. by wet granulation, dry granulation or direct compression. For example, dry granulation procedures include mixing the solid excipients (except lubricants), compacting the mixture in a compactor (e.g. a roller compactor) or double compression, milling the compacted mass, screening the milled granules, mixing with a lubricant and compressing the mixture into tablets. Direct compression procedures generally comprise mixing the solid excipients in one or more stages and compressing the uniform mixture into tablets. After tablet formation, the tablets may optionally be coated with the controlled release coating discussed hereinabove. The tablets can be of any size and shape. Tablet excipients can also be included and may be, for example, diluents, retardants, and lubricants, as well as other pharmaceutically acceptable excipients. These excipients may be present in the core region and/or any other region of the formulation. Generally, diluents according to the present invention are inert materials that can be added to the active ingredient to make them more acceptable. Diluents are fillers designed to make up the required bulk of the tablet when the drug dosage itself is inadequate to produce this bulk. Tablet formulations may contain diluents for secondary reasons, such as to provide better tablet properties such as improved cohesion, to permit use of direct compression manufacturing, or to promote flow.

Lactose is the most widely used diluent in tablet formulation. Lactose is an excipient that has no reaction with most drugs, whether it is used in the hydrous or anhydrous form. Anhydrous lactose has the advantage over lactose in that it does not undergo the Maillard reaction, which can lead to browning and discoloration with certain drugs. Lactose formulations show good drug release rates, their granulations are readily dried and the tablet disintegration times of lactose tablets are not strongly sensitive to variations in tablet hardness. Usually fine grades of lactose are used in the preparation of tablets because the fine size permits better mixing with other formulation ingredients and utilizes the binder more efficiently. Generally, the grade of lactose chosen is dependent on the type of dosage form being developed. Direct-compression grades are often used to carry small quantities of drug and this permits tablets to be made without granulating.

Direct-compression grades of lactose are more fluid and more compressible than crystalline or powdered lactose, and generally, they are composed of spray-dried lactoses, which contain specially prepared pure alpha lactose monohydrate along with a small amount of amorphous lactose. The amorphous lactose improves the compression force/hardness profile of the lactose. Other specially produced direct-compression grades of lactose do not contain amorphous material but may contain glassy or vitreous areas which impart improved compressibility. The use of direct-compression grades of lactose results in tablets of higher breaking strength than standard lactose. Concentrations of lactose generally used in these formulations are from about 65 to about 85%.

Anhydrous dibasic calcium phosphate is used in pharmaceutical products because of its compaction properties, and the good-flow properties of the coarse grade material. The predominant deformation mechanism of anhydrous dibasic calcium phosphate coarse-grade is brittle fracture and this reduces the strain sensitivity of the material, thus allowing easier transition from the laboratory to production scale. Anhydrous dibasic calcium phosphate is abrasive and a lubricant is required for tabletting, for example, about 1% magnesium stearate or about 1% sodium stearyl fumarate. Two particle size grades of anhydrous dibasic calcium phosphate are used in the pharmaceutical industry. Milled material is typically used in wet-granulated or roller-compacted formulations. The unmilled or coarse-grade material is typically used in direct-compression formulations. Anhydrous dibasic calcium phosphate is non-hygroscopic and stable at room temperature it does not hydrate to form the dihydrate.

Retardants control the release of an active substance from a tablet matrix. As mentioned above, retardation of drug release may be achieved either by a controlled release coating or by embedding the drug in a controlled release matrix of a hydrophilic or hydrophobic polymer or a combination thereof.

Pregelatinized starch (available as Starch 1500® from Colorcon® of West Point, Pa.), according to the present invention, influences the drug release from the hydrophilic sustained release matrix formulations. Use of Starch 1500® significantly reduces the drug release as compared to formulations containing microcrystalline cellulose (MCC) or lactose. Starch 1500® is not an inert filler in HPMC matrices, but it actively contributes to the mechanism of drug release causing a decrease in drug release rate. Increasing concentrations of Starch 1500® (about 20, about 35 and about 49.25% w/w) in the formulations caused a decrease in their release profiles.

In a preferred embodiment of the present invention, the extended release pharmaceutical formulation of tizanidine is uniformly dispersed in a matrix including at least hydroxypropyl methyl cellulose and partially or fully pregelatinized starch.

Microcrystalline cellulose (available as Avicel® from FMC Corporation of Philadelphia, Pa.), according to the present invention, is a direct compression material. Due to the self disintegrating property of Avicel® it requires little lubricant. Microcrystalline cellulose is often added to tablet formulation for several possible functions. It is a commonly employed excipient. The present invention employs Avicel® pH-102 in the concentration of about 20 to about 90% by weight, based on the total weight of the composition. In addition to its use as a binder/diluent, microcrystalline cellulose also has some lubricant properties.

Lubricants reduce friction by interposing a film of low shear strength between the tablet mass and the confining die wall interface during tablet formation and ejection. They also play the role of anti-adherents wherein they prevent sticking to surfaces like the faces of tablet punches. Lubricants also act as glidants thereby improving the flow by modifying the interaction between particles. Therefore, the concept of a lubricant system is generally the use of two substances to maximize overall lubricant effect in all three areas as lubricant, antiadherent and glidant, for example, combining magnesium stearate with colloidal silica.

Stearic acid, according to the present invention, acts as a lubricant. The amount of stearic acid acts can range from about 1 to about 3% by weight, based on the total weight of the composition.

Glidants for use herein include, but are not limited to, colloidal silicon dioxide (available as Aerosil® from Degussa AG of Dusseldorf, Genmany) Generally, glidants can be used in a concentration of about 0.1 to about 0.5% by weight, based on the total weight of the composition.

The pharmaceutical formulation of the present invention may contain other optional ingredients that are also typically used in pharmaceuticals such as, for example, coloring agents, preservatives, flavorings, and the like.

The following examples are provided to enable one skilled in the art to practice the invention and are merely illustrative of the invention. The examples should not be read as limiting the scope of the invention as defined in the claims.

### EXAMPLE 1

All ingredients except stearic acid were sifted through mesh # 30. The ingredients as set forth below in Table 1 were blended together by geometric dilution and mixed thoroughly in a double-cone blender and then lubricated with stearic acid that was previously passed through mesh # 60. The blend was directly compressed into tablets having a target weight of about 300 mg.

TABLE 1

| Ingredient | Quantity/tab (mg) | % w/w |
|---|---|---|
| Tizanidine HCl | 6.864 | 2.29 |
| Starch 1500 | 141.04 | 47.01 |
| HPMC K100M CR | 150 | 50.00 |
| Colloidal SiO2 | 0.6 | 0.20 |
| Stearic acid | 1.5 | 0.50 |
| Average Tablet Weight (mg) | 300 | |

The tablets were then tested in a VanKel dissolution bath (USP apparatus 2, 50 rpm) at 37° C. in 500 ml of 0.01(N)HCl for 16 hours. The tizanidine in the samples was determined by an HPLC system on a C-18 column using an aqueous buffer pH 7.4: methanol with UV detection at 230 nm. The results set forth below in Table 2.

TABLE 2
Dissolution profile

| Time (hours) | % tizanidine release |
|---|---|
| 0 | 0 |
| 1 | 20 |
| 3 | 39 |
| 4.5 | 50 |
| 6 | 59 |
| 8 | 69 |
| 10 | 76 |
| 14 | 88 |
| 16 | 93 |

### EXAMPLE 2

The ingredients set forth below in Table 3 were sifted through mesh # 30 and mixed in a planetary mixer. The blend was then granulated using Eudragit® NE 30D dispersion (134 g of the dispersion containing 40 g of total solid content). The granules were dried to obtain a loss-on-drying (LOD) value below 2% and then milled. The granules were then passed through mesh # 20 and lubricated with stearic acid in a double-cone blender. The blend was compressed into tablets having target weight of about 300 mg.

TABLE 3

| Ingredient | Quantity/tab (mg) | % w/w |
|---|---|---|
| Tizanidine HCl | 6.864 | 2.29 |
| Lactose, anhydrous | 40.64 | 13.55 |
| Starch 1500 | 61 | 20.33 |
| HPMC K100M CR | 150 | 50.00 |
| Eudragit NE 30D | 134 (40) | 13.33 |
| Stearic acid | 1.5 | 0.50 |
| Average Tablet Weight (mg) | 300 | |

In Vitro Dissolution Profile

The tablets were then tested in a similar manner as discussed in Example 1. The results set forth below in Table 4.

TABLE 4
Dissolution profile

| Time (hours) | % tizanidine release |
|---|---|
| 0 | 0 |
| 1 | 21 |
| 3 | 42 |
| 4.5 | 52 |
| 6 | 60 |
| 8 | 69 |
| 10 | 76 |
| 14 | 85 |
| 16 | 89 |

### EXAMPLE 3

All ingredients except stearic acid were sifted through mesh # 30, mixed thoroughly in a double-cone blender and then lubricated with stearic acid that was previously passed through mesh # 60. The blend was directly compressed into tablets having a target weight of about 300 mg as set forth below in Table 5.

TABLE 5

| Ingredient | Quantity/tab (mg) | % w/w |
|---|---|---|
| Tizanidine HCl | 6.864 | 2.29 |
| Lactose, anhydrous | 30 | 10.00 |
| Starch 1500 | 81.04 | 27.01 |
| HPMC K100M CR | 180 | 60.00 |
| Colloidal SiO2 | 0.6 | 0.20 |
| Stearic acid | 1.5 | 0.50 |
| Average Tablet Weight (mg) | 300 | |

In Vitro Dissolution Profile

The tablets were then tested in a similar manner as discussed in Example 1. The results set forth below in Table 6.

TABLE 6
Dissolution profile

| Time (hours) | % tizanidine release |
|---|---|
| 0 | 0 |
| 1 | 22 |
| 3 | 42 |
| 4.5 | 54 |
| 6 | 63 |
| 8 | 74 |
| 10 | 82 |
| 14 | 94 |
| 16 | 99 |

### EXAMPLE 4

Tizanidine HCl and 5% w/v of Ethocel® were sieved through mesh # 40 and dissolved with stirring in ethanol 95% to give a slightly gel-like mass. The remaining amount of Ethocel® was added to a planetary mixer along with starch 1500 and about 30% w/w of HPMC K100M and dry-mixed for 5 minutes and then granulated with the gel-like mass of tizanidine HCl and Ethocel® obtained earlier. The granules were dried to obtain a loss-on-drying value below 2%, milled and passed through mesh # 30. These granules were then blended with the remaining (20% w/w) of HPMC K100M and colloidal SiO₂ in a double-cone blender and lubricated with stearic acid that was previously passed through mesh # 60. The final blend was compressed into tablets having target weight of about 300 mg as set forth below in Table 7.

TABLE 7

| Ingredient | Quantity/tab (mg) | % w/w |
|---|---|---|
| Tizanidine HCl | 6.864 | 2.29 |
| Starch 1500 | 81.04 | 27.01 |
| HPMC K100M CR | 150 | 50.00 |
| Ethyl cellulose STD FP 100 | 60 | 20.00 |
| (Ethocel) | | |
| Colloidal SiO2 | 0.6 | 0.20 |
| Stearic acid | 1.5 | 0.50 |
| Average Tablet Weight (mg) | 300 | |

The tablets were then tested in a similar manner as discussed in Example 1. The results set forth below in Table 8.

TABLE 8
Dissolution profile

| Time (hours) | % tizanidine release |
|---|---|
| 0 | 0 |
| 1 | 14 |
| 3 | 30 |
| 4.5 | 38 |
| 6 | 45 |
| 8 | 53 |
| 10 | 60 |
| 14 | 69 |
| 16 | 74 |

Tizanidine Extended Release has shown relative bioavailability of 63% (as reflected by AUC_{(0-∞)}, Area Under Plasma Concentration vs. time curve) with sustained levels of drug appearing up to 24 hours. A shift in peak time from 1.40 hours (for the Tizanidine IR product) to 4.10 hours (for Extended Release product) and a two hour extension in half-life with respect to the Tizanidine IR product were supportive of sustained drug release from the product of present invention without any signs of dose dumping. In addition the extended Release formulation has shown MRT (Mean Residence Time) of 8.89 hours vs. 2.84 hours of the Tizanidine IR product reflecting the continuous presence of active drug levels during the dosage period.

TABLE 9
Summary PK of Tizanidine (n = 10)

| | | C^{∞}_{avg} | AUC₍₀₋ₜ₎ | AUC_{(0-∞)} | | |
|---|---|---|---|---|---|---|
| Mean Data | Cₘₐₓ (ng/ml) | (ng/ml) | (ng · hr/ml) | (ng · hr/ml) | t_{1/2} (hr) | Tₘₐₓ (hr) |
| Reference | 5.45 | 1.06 | 41.25 | 42.95 | 2.25 | 1.40 |
| (IR Product) | | | | | | |
| Test | 3.28 | 1.40 | 24.85 | 26.79 | 4.15 | 4.10 |
| (Extended | | | | | | |
| Release | | | | | | |
| Formulation) | | | | | | |
| Ratio (%) | 60.18 | 132.08 | 60.24 | 62.37 | 184.44 | 292.86 |

In considering the above single day data for both the Tizanidine IR and Extended Release formulations, the predicted PK parameters at doses given for 5 day chronic treatment (i.e., for the Tizanidine IR formulation the dose design is 2 mg t.i.d. for 5 days and for Extended Release formulation the dose design is 6 mg o.d. for 5 days) are tabulated below. The Extended Release formulation is found to be 100% equivalent with respect to the Tizanidine IR product.

TABLE 10
Predicted Summary PK of Tizanidine (n = 10)
after 5 day chronic treatment

| | C^{∞}_{avg} | AUC₍₀₋ₜ₎ | AUC_{(0-∞)} | | |
|---|---|---|---|---|---|
| Mean Data | (ng/ml) | (ng · hr/ml) | (ng · hr/ml) | T_{1/2} (hr) | MRT (hr) |
| Reference | 1.06 | 130.51 | 130.73 | 2.25 | 2.84 |
| (IR Product) | | | | | |
| Test | 1.08 | 128.29 | 129.49 | 4.15 | 8.89 |
| (Extended- | | | | | |
| Release | | | | | |
| Formulation) | | | | | |
| Ratio (%) | 101.89 | 98.30 | 99.05 | 184.14 | 313.03 |

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

1. An oral extended-release pharmaceutical dosage form comprising (a) a therapeutically effective amount of an active ingredient comprising tizanidine or a pharmaceutically acceptable salt or ester thereof; and, (b) from about 30 to about 70 percent by weight of a pharmaceutically acceptable hydrophilic and/or hydrophobic matrix for the extended release of the active ingredient, and suitable amounts of one or more therapeutically inert, pharmaceutically acceptable adjunct materials.

2. The oral extended-release pharmaceutical dosage form of claim 1, wherein the matrix comprises a hydrophilic gel-forming cellulosic polymer

3. The oral extended-release pharmaceutical dosage form of claim 1, wherein the matrix is a hydrophilic matrix comprising a hydrophilic gel-forming polymer selected from the group consisting of ethyl cellulose, methyl cellulose, hydroxypropyl cellulose, hydroxyethyl cellulose, sodium carboxymethyl cellulose, hydroxypropyl methylcellulose and mixtures thereof.

4. The oral extended-release pharmaceutical dosage form of claim 1, wherein the matrix comprises a hydrophilic gel-forming polymer, the hydrophilic gel-forming polymer being at least a hydroxypropyl methylcellulose having a number average molecular weight of at least about 20,000.

5. The oral extended-release pharmaceutical dosage form of claim 4, wherein the hydroxypropyl methylcellulose has a hydroxypropoxyl substitution of from about 7 to about 12 weight percent and a methoxyl substitution of from about 19 to about 24 weight percent.

6. The oral extended-release pharmaceutical dosage form of claim 1, wherein the hydrophilic matrix comprises a hydrophilic polymer selected from the group consisting of cellulose ether, cellulose esters, acrylic acid polymers, polyethylene oxides and mixtures thereof.

7. The oral extended-release pharmaceutical dosage form of claim 1, wherein the matrix is a mixture of the hydrophilic and hydrophobic matrix components.

8. The oral extended-release pharmaceutical dosage form of claim 7, wherein the hydrophobic matrix comprises a component selected from the group consisting of a hydrophobic polymer, a hydrophobizing agent and mixtures thereof.

9. The oral extended-release pharmaceutical dosage form of claim 1, wherein the matrix component is present in an amount of from about 40 to about 65 percent by weight.

10. The oral extended-release pharmaceutical dosage form of claim 4, wherein the matrix components is present in an amount of from about 40 to about 65 percent by weight.

11. The oral extended-release pharmaceutical dosage form of claim 1, wherein the therapeutically inert, pharmaceutically acceptable adjunct materials are selected from the group consisting of a starch, a lactose and mixtures thereof.

12. The oral extended-release pharmaceutical dosage form of claim 1, wherein the dosage form is in the form of granules, pellets, or a powder blend.

13. The oral extended-release pharmaceutical dosage form of claim 12, wherein a portion of the dosage form in the form of granules, pellets, or a powder blend is in the form of granules, pellets, or a powder blend for immediate release of the active ingredient.

14. The oral extended-release pharmaceutical dosage form of claim 1, wherein the dosage form is in the form of enteric coated pellets or enteric coated granules.

15. The oral extended-release pharmaceutical dosage form of claim 1, wherein the dosage form is in the form of a tablet.

16. The oral extended-release pharmaceutical dosage form of claim 1, wherein the dosage form is in the form of an enteric coated tablet.

17. The oral extended-release pharmaceutical dosage form of claim 1, wherein the coating of the enteric coated tablet comprises a material selected from the group consisting of a water-insoluble wax, a water-insoluble cellulose, a water-insoluble polymer, and combinations thereof.

18. The oral extended-release pharmaceutical dosage form of claim 17, wherein the water-insoluble cellulose comprises ethyl cellulose.

19. The oral extended-release pharmaceutical dosage form of claim 17, wherein the water-insoluble polymer comprises an acrylic resin.

20. The oral extended-release pharmaceutical dosage form of claim 19, wherein the acrylic resin is a poly(meth)acrylate copolymer.

21. The oral extended-release pharmaceutical dosage form of claim 17, wherein the coating further comprises a water-soluble polymer.

22. The oral extended-release pharmaceutical dosage form of claim 21, wherein the water-soluble polymer is a polyvinyl pyrrolidine.

23. The oral extended-release pharmaceutical dosage form of claim 17, wherein the coating further comprises a hydrophilic pore former.

24. The oral extended-release pharmaceutical dosage form of claim 23, wherein the hydrophilic pore former is selected from the group consisting of sodium chloride, mannitol and mixtures thereof.

25. The oral extended-release pharmaceutical dosage form of claim 17, wherein the coating further comprises a plasticizer.

26. The oral extended-release pharmaceutical dosage form of claim 1, further comprising one or more pharmaceutically acceptable excipients.

27. The oral extended-release pharmaceutical dosage form of claim 26, wherein the excipient is selected from the group consisting of a diluent, a retardant, a lubricant, a glidant and mixtures thereof.

28. The oral extended-release pharmaceutical dosage form of claim 26, wherein the excipient is microcrystalline cellulose.

29. The oral extended-release pharmaceutical dosage form of claim 27, wherein the diluent is a lactose selected from the group consisting of hydrous lactose, anhydrous lactose, crystalline lactose, powdered lactose and mixtures thereof.

30. The oral extended-release pharmaceutical dosage form of claim 26, wherein the excipient is anhydrous dibasic calcium phosphate.

31. The oral extended-release pharmaceutical dosage form of claim 27, wherein the lubricant is selected from the group consisting of magnesium stearate, sodium stearyl fumarate, stearic acid and mixtures thereof.

32. The oral extended-release pharmaceutical dosage form of claim 15, wherein the dosage form is a once daily dose tablet.

33. The oral extended-release pharmaceutical dosage form of claim 12, wherein the dosage form is a once daily dose capsule.

34. A capsule comprising the granules, pellets, or a powder blend of claim 12.

35. A capsule comprising the granules, pellets, or a powder blend of claim 13.

36. A capsule comprising the enteric coated granules or enteric coated pellets of claim 14.

37. The oral extended-release pharmaceutical dosage form of claim 1, wherein the release period is from about 8 to about 16 hours.

38. The oral extended-release pharmaceutical dosage form of claim 1, wherein the release period is from about 14 to about 16 hours.

39. An extended-release pharmaceutical dosage form comprising:
(a) a core region comprising a therapeutically effective amount of an active ingredient comprising one or more alpha-2 adrenergic agonists or a pharmaceutically acceptable salt or ester thereof;
(b) a matrix material integrated with the core region; and
(c) a controlled release coating on the core region for the extended release of the active ingredient.

40. The extended-release pharmaceutical dosage form of claim 39, wherein the alpha-2 adrenergic agonist is tizanidine.

41. The extended-release pharmaceutical dosage form of claim 39, wherein the controlled release coating comprises a material selected from the group consisting of a water-insoluble wax, a water-insoluble cellulose, a water-insoluble polymer and combinations thereof.

42. The extended-release pharmaceutical dosage form of claim 41, wherein the water-insoluble cellulose is ethyl cellulose.

43. The extended-release pharmaceutical dosage form of claim 41, wherein the water-insoluble polymer comprises an acrylic resin.

44. The extended-release pharmaceutical dosage form of claim 43, wherein the acrylic resin is a poly(meth)acrylate copolymer.

45. The extended-release pharmaceutical dosage form of claim 41, wherein the controlled release coating further comprises a water-soluble polymer.

46. The extended-release pharmaceutical dosage form of claim 45, wherein the water-soluble polymer is a polyvinyl pyrrolidine.

47. The extended-release pharmaceutical dosage form of claim 41, wherein the controlled release coating further comprises a water-soluble cellulose.

48. The extended-release pharmaceutical dosage form of claim 47, wherein the water-soluble cellulose is hydroxypropyl methylcellulose or hydroxypropyl cellulose.

49. The extended-release pharmaceutical dosage form of claim 41, wherein the controlled release coating further comprises a hydrophilic pore former.

50. The extended-release pharmaceutical dosage form of claim 49, wherein the hydrophilic pore former is selected from the group consisting of sodium chloride, mannitol and mixtures thereof.

51. The extended-release pharmaceutical dosage form of claim 41, wherein the controlled release coating further comprises a plasticizer.

52. The extended-release pharmaceutical dosage form of claim 39, further comprising one or more pharmaceutically acceptable excipients.

53. The extended-release pharmaceutical dosage form of claim 39, wherein the matrix material comprises at least one material selected from the group consisting of a hydrophilic polymer, a hydrophobic polymer, a hydrophobic wax, a hydrophobic fat, a hydrophobic long-chain fatty acid, a hydrophobic fatty alcohol, derivatives thereof and mixtures thereof.

54. The extended-release pharmaceutical dosage form of claim 53, wherein the hydrophilic polymer is selected from the group consisting of cellulose ether, cellulose esters, acrylic acid polymers, polyethylene oxides and mixtures thereof.

55. The extended-release pharmaceutical dosage form of claim 39, wherein the matrix material is a hydrophilic matrix comprising a hydrophilic gel-forming polymer selected from the group consisting of ethyl cellulose, methyl cellulose, hydroxypropyl cellulose, hydroxyethyl cellulose, sodium carboxymethyl cellulose, hydroxypropyl methylcellulose and mixtures thereof.

56. The extended-release pharmaceutical dosage form of claim 39, wherein the matrix material comprises a hydroxypropyl methylcellulose having a number average molecular weight of at least about 20,000.

57. The extended-release pharmaceutical dosage form of claim 56, wherein the hydroxypropyl methylcellulose has a hydroxypropoxyl substitution of from about 7 to about 12 weight percent and a methoxyl substitution of from about 19 to about 24 weight percent.

58. The extended-release pharmaceutical dosage form of claim 53, further comprising one or more pharmaceutically acceptable excipients.

59. The extended-release pharmaceutical dosage form of claim 58, wherein the excipient is selected from the group consisting a diluent, a retardant, a lubricant, a glidant and mixtures thereof.

60. The extended-release pharmaceutical dosage form of claim 58, wherein the excipient is microcrystalline cellulose.

61. The extended-release pharmaceutical dosage form of claim 59, wherein the diluent is lactose.

62. The extended-release pharmaceutical dosage form of claim 61, wherein the lactose is selected from the group consisting of hydrous lactose, anhydrous lactose, crystalline lactose, powdered lactose and mixtures thereof.

63. The extended-release pharmaceutical dosage form of claim 59, wherein the lubricant is selected from the group consisting of magnesium stearate, sodium stearyl fumarate, stearic acid and mixtures thereof.

64. The extended-release pharmaceutical dosage form of claim 39, wherein the matrix material further comprises a pregelatinized starch.

65. The extended-release pharmaceutical dosage form of claim 39, wherein the dosage form is in the form of granules, pellets, or a powder blend.

66. The extended-release pharmaceutical dosage form of claim 65, wherein a portion of the dosage form in the form of granules, pellets, or a powder blend is in the form of granules, pellets, or a powder blend for immediate release of the active ingredient.

67. The extended-release pharmaceutical dosage form of claim 39, wherein the dosage form is in the form of enteric coated pellets or enteric coated granules.

68. The extended-release pharmaceutical dosage form of claim 39, wherein the dosage form is in the form of a tablet.

69. The extended-release pharmaceutical dosage form of claim 68, wherein the dosage form is a once daily dose tablet.

70. The extended-release pharmaceutical dosage form of claim 65, wherein the dosage form is a once daily dose capsule.

71. A capsule comprising the granules, pellets, or a powder blend of claim 65.

72. A capsule comprising the granules, pellets, or a powder blend of claim 66.

73. A capsule comprising the enteric coated granules or enteric coated pellets of claim 67.

74. The extended-release pharmaceutical dosage form of claim 39, wherein the release period is from about 8 to about 16 hours.

75. The extended-release pharmaceutical dosage form of claim 39, wherein the release period is from about 14 to about 16 hours.

76. A method for the treatment or prevention of spasticity in a subject is provided comprising administering to the subject in need of such treatment or prevention a therapeutically effective amount of a once-a-day extended release pharmaceutical formulation according to claim 1.

77. A method for the treatment or prevention of spasticity in a subject is provided comprising administering to the subject in need of such treatment or prevention a therapeutically effective amount of a once-a-day extended release pharmaceutical formulation according to claim 39.

78. A process for the preparation of an extended release pharmaceutical dosage form comprising:
(a) preparing a core comprising a therapeutically effective amount of an active ingredient comprising one or more alpha-2 adrenergic agonists or a pharmaceutically acceptable salt or ester thereof;
(b) integrating a hydrophilic matrix material with the core; and
(c) coating the core with a controlled release coating that extends the release of the active ingredient.

79. The process of claim 78, wherein the alpha-2 adrenergic agonist is tizanidine.

80. The process of claim 78, wherein hydrophilic matrix comprises a hydrophilic gel-forming polymer selected from the group consisting of ethyl cellulose, methyl cellulose, hydroxypropyl cellulose, hydroxyethyl cellulose, sodium carboxymethyl cellulose, hydroxypropyl methylcellulose and mixtures thereof.

81. The process of claim 78, wherein the hydrophilic matrix comprises a hydrophilic gel-forming polymer, the hydrophillic gel-forming polymer being at least a hydroxypropyl methylcellulose having a number average molecular weight of at least about 20,000.

82. The process of claim 81, wherein the hydroxypropyl methylcellulose has a hydroxypropoxyl substitution of from about 7 to about 12 weight percent and a methoxyl substitution of from about 19 to about 24 weight percent.

83. The process of claim 78, wherein the hydrophilic matrix comprises a hydrophilic polymer selected from the group consisting of cellulose ether, cellulose esters, acrylic acid polymers, polyethylene oxides and mixtures thereof.

84. The process of claim 78, wherein the hydrophilic matrix is a mixture of the hydrophilic and a hydrophobic matrix component.

85. The process of claim 84, wherein the hydrophobic matrix comprises a component selected from the group consisting of a hydrophobic polymer, a hydrophobizing agent and mixtures thereof.

86. The process of claim 78, wherein the matrix component is present in an amount of from about 40 to about 65 percent by weight.

87. The process of claim 81, wherein the matrix component is present in an amount of from about 40 to about 65 percent by weight.

88. The process of claim 78, wherein the dosage form is in the form of granules, pellets, or a powder blend.

89. The process of claim 78, wherein a portion of the dosage form is in the form of granules, pellets, or a powder blend is in the form of granules, pellets, or a powder blend for immediate release of the active ingredient.

90. The process of claim 78, wherein the dosage form is in the form of enteric coated pellets or enteric coated granules.

91. The process of claim 78, wherein the dosage form is in the form of a tablet.

92. The process of claim 78, wherein the controlled release coating comprises a material selected from the group consisting of a water-insoluble wax, a water-insoluble cellulose, a water-insoluble polymer and combinations thereof.

93. The process of claim 92, wherein the water-insoluble cellulose comprises ethyl cellulose.

94. The process of claim 92, wherein the water-insoluble polymer comprises an acrylic resin.

95. The process of claim 94, wherein the acrylic resin is a poly(meth)acrylate copolymer.

96. The process of claim 92, wherein the coating further comprises a water-soluble polymer.

97. The process of claim 96, wherein the water-soluble polymer is a polyvinyl pyrrolidine.

98. An orally administrable extended release dosage form comprising a core comprised of an active pharmaceutical ingredient comprising tizanidine or a pharmaceutically acceptable salt or ester thereof, wherein said dosage form provides once daily dosing for therapeutic relief from skeletal muscle spasms.
